(19) United States
(12) Patent Application Publication
(10) Pub. No.: US 2006/0254604 A1
(43) Pub. Date: Nov. 16, 2006

(54) Simulated cigarette which is designed to help quell tobacco addiction

(76) Inventor: Jose Antonio Martinez Fernandez, Pontevedra (ES)

Correspondence Address: EGBERT LAW OFFICES, 412 MAIN STREET, 7TH FLOOR, HOUSTON, TX, 77002, US

(21) Appl. No.: 10/563,419

(22) PCT Filed: Jul. 21, 2004
(86) PCT No.: PCT/ES04/00342

§ 371 Date: May 11, 2006

(30) Foreign Application Priority Data
Jul. 24, 2003 (ES) U 200301783

(51) Int. Cl. A61M 15/06

(52) U.S. Cl. 131/273; 131/270; 128/202.21

(57) ABSTRACT

The invention relates to a simulated cigarette to help quell tobacco addiction. The inventive simulated cigarette includes a cylindrical body which is made from a plastic material. The aforementioned cylindrical body contains an inner plug close to one of the ends thereof, natural essences which are disposed next to a filter, and a secondary filter having an extended inner diameter, to which a tip is connected.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention is applicable in the industry dedicated to manufacturing auxiliary elements to reduce drug addiction habits.

### BACKGROUND OF THE INVENTION

The applicant is aware of the great difficulty that any habitual tobacco consumer has when attempting to quit this habit which creates tobacco addiction.

In the attempt to aid tobacco consumers, there are currently different chemical products which, presented as patches or as pills, attempt to aid the consumer to lose the state of addiction which he or she is in.

However like others, the applicant has found that these chemical products only partially comply with or cover the task, since it has been shown that the overwhelming majority of smokers not only crave the direct effects of tobacco on their system due to the lack of consumption, but they greatly crave and miss the mechanics of the act of smoking.

Accordingly, the applicant is not aware of the current existence of an invention that is able to reduce the habit of tobacco consumption.

### BRIEF SUMMARY OF THE INVENTION

The present specification refers to a simulated cigarette which is designed to help quell tobacco addiction, the evident purpose of which is to eliminate, reduce or at least palliate addiction to tobacco, being made up of a body similar to a standard cigarette and which, when inhaling through it has a small resistance to air, similar to the resistance created from the inhalation carried out with a standard cigarette and at the same time and in a parallel manner, it provides the consumer with a fresh and pleasant taste that decreases or reduces anxiety resulting from a lack of nicotine.

### BRIEF DESCRIPTION OF THE SEVERAL VIEWS OF THE DRAWINGS

To complement the description being made and for the purpose of aiding to better understand the features of the invention, a set of drawings is attached to the present specification as an integral part thereof, in which the following is shown with an illustrative and non-limiting character.

FIG. 1 shows a perspective view of the object of the invention corresponding to a simulated cigarette to help quell tobacco addiction.

FIG. 2 shows an exploded perspective view of the body of the cigarette, the tip and plug forming the object shown in FIG. 1.

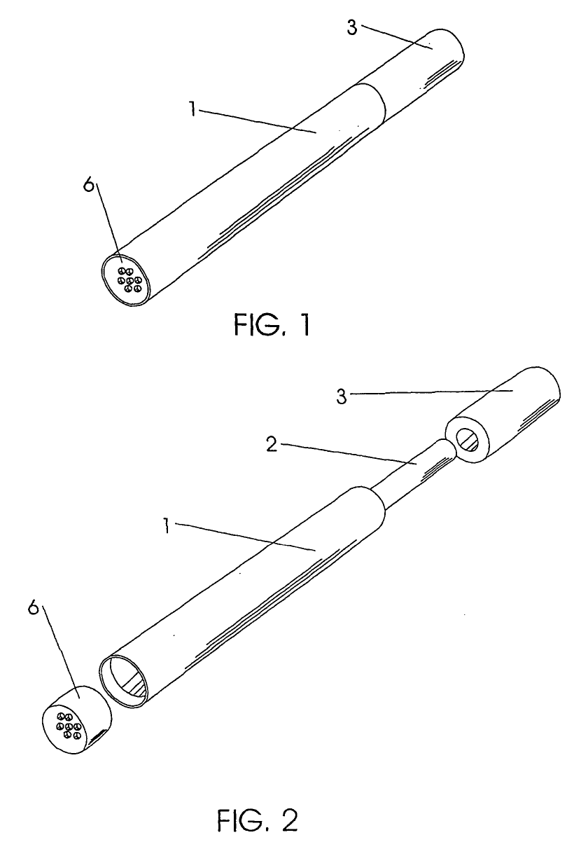

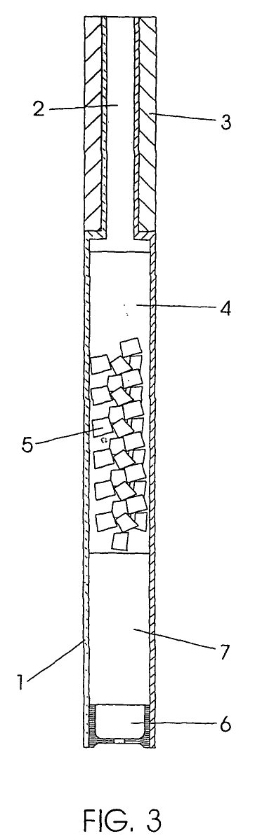

Finally FIG. 3 shows a sectional view of the object shown in FIG. 1, this graphic representation showing the arrangement of the different components forming it.

### DETAILED DESCRIPTION OF THE INVENTION

The simulated cigarette which is designed to help quell tobacco addiction proposed by the invention is configured as a highly important element or product as it combines therein the effects demanded from the simulated cigarette, since on one hand it allows maintaining said manual mechanics that is a habit for smokers, while on the other hand it replaces tobacco smoke due to the inhalation of the duly combined essences generated from the simulated cigarette, which eliminate or quell a smoker's craving.

More specifically, the simulated cigarette which is designed to help quell tobacco addiction is made up of a tubular or cylindrical body made from a rigid material, presented in the form of a tube in which two areas with different diameters are arranged, the one with the larger diameter, and in turn longest in length, having the purpose of being used as an internal housing for the tobacco replacement products, whereas the area having a smaller diameter is intended for externally housing a tip made of a soft material which is both visually and mechanically equivalent to the tip of a conventional cigarette, this assembly configured by means of the body, tip and their outer features adopting the general shape of a conventional cigarette.

A filter is arranged in the area having the larger diameter of said body forming the simulated cigarette, after its inner end, with the function of achieving that the person who inhales obtains the essences and the flavor of the products existing inside the recipient where they are housed, thus preventing the product form reaching the consumer in solid form, effecting and assuring the fact of being presented as a product lasting between one and two weeks, and then the product is added, which is made up of a menthol part assuring freshness upon inhalation, valerian, which reduces anxiety created by the lack of nicotine, and then the remaining elements making up the cigarette can have different flavors, such as mint, strawberry, etc., so that the consumer will always have the flavor he or she most likes in the simulated cigarette, and therefore making it more comfortable to replace tobacco.

### PREFERRED EMBODIMENT OF THE INVENTION

In view of these figures it can be seen how the simulated cigarette which is designed to help quell tobacco addiction is made up of a tubular body (1) made from a rigid material such as a plastic material, in which two areas having different diameters are arranged, one having a larger diameter and a greater axial height which is configured as an extension of the body (1) and is marked with reference number (2), forming a neck intended for externally receiving a tip (3) located on its outer end, made from a soft material, with an axial height coinciding with the axial height of the area (2) and an outer diameter in turn coinciding with the general body (1), such that after assembling the tip (3) on the general body, this assembly has a configuration and dimensions similar to those of a standard cigarette with a tip.

Arranged inside the body (1), specifically in the central area thereof, there is a filter (4) allowing the passage of air through it, retaining the remaining components of the simulated cigarette, specifically the product (5) made up of a mixture of menthol, valerian and a flavor chosen for this unit, and closing the assembly of the area (1) by means of a plug (6) made from a rigid material, provided with perforations allowing the passage of air through it.

The incorporation of a filter (4) has been provided in the area (1) opposite to the area provided with the plug (6) for the purpose of achieving that the mixture carried out with menthol, valerian and the flavoring agent forming the essences (5) is open so that the components in question perform the appropriate modification of volumes so as to obtain a stronger or smoother flavor of the simulated cigarette.

I claim:

1. A simulated cigarette to help quell tobacco addiction, comprising:
a tubular body being comprised of a rigid material, preferably plastic;
a plug, being incorporated in an inner area of said tubular body and specifically at one end of said tubular body, and being located adjacent to a first filter having a central hollow space where natural essences are incorporated; and
a second filter arranged on an end adjacent to the end provided with the first filter, having an extension in an outer area thereof with a smaller diameter and length on which a flexible tip is coupled next to said first filter, said second filter having an extended inner diameter, to which said flexible tip is connected.
